(19) United States
(12) Patent Application Publication
(10) Pub. No.: US 2020/0347448 A1
(43) Pub. Date: Nov. 5, 2020

(54) FUNCTIONAL METAGENOMICS

(71) Applicant: WILD BIOTECH, Rehovet (IL)

(72) Inventors: Ido Bachelet, Tel Aviv-Yafo (IL); Moran Avraham Kopel, Pardes Flana (IL); Liron Anna Bassali, Tel Aviv (IL); Neta Raab, Flod Flasharon (IL)

(21) Appl. No.: 16/758,684

(22) PCT Filed: Oct. 23, 2018
(86) PCT No.: PCT/IB2018/001312

§ 371 Date: Apr. 23, 2020

Related U.S. Application Data
(60) Provisional application No. 62/575,841, filed on Oct. 23, 2017.

(51) Int. Cl. C12Q 1/6869; C12Q 1/686

(52) U.S. Cl. CPC C12Q 1/6869; C12Q 2563/185; C12Q 1/686

(57) ABSTRACT

The present disclosure describes methods for identifying a microbe that modulates an activity of a target cell, a target virus, or an enzymatic reaction of a target substrate.

### RELATED APPLICATIONS

This application claims the benefit of priority to U.S. Provisional Patent Application Ser. No. 62/575,841, filed Oct. 23, 2017, which is hereby incorporated by reference in its entirety.

### BACKGROUND

Organisms survive in their natural habitats based on adaptations that are encoded, in part, by their microbiomes (e.g., in/on their gut, skin, roots, saliva, leaves, etc.). Isolation and identification functionally relevant bacteria from such microbial populations would allow microbiomes to be harnessed in novel ways, including to neutralize toxins, inhibit the growth of harmful pathogens (e.g., pathogenic bacteria and fungi).

Functional metagenomics is a powerful experimental approach for studying the function of diverse population of organisms though the analysis of extracted DNA of mixed microbial populations. Due to the steady improvement in sequencing technologies over the past several years, generating large amounts of sequence is becoming routine. However, it remains difficult to link specific microbial phyla with specific functions performed by the microbiome.

Thus, new methods and compositions for identifying from a microbiome specific bacterial species and/or strains that possess desirable features using functional metagenomic approaches are needed.

### SUMMARY

In certain aspects, the present disclosure relates to methods for performing functional metagenomics analysis of microbiome populations by distributing individual bacterium from the microbiome into droplets or microwells and then testing their interactions with predetermined targets. The individual bacterium are allowed to interact with targets, following which the entire emulsion is analyzed by flow cytometry, sorting, microscopy, spectrophotometry, fluorometery, PCR, or next-generation sequencing (e.g., whole-genome sequencing, RNA-seq, etc.), in order to identify strains that have a desired effect on the target (e.g. inhibition of target cell growth, inducement of target cell killing, cleavage of a target peptide substrate, etc.)

In one aspect, provided herein is a method for identifying a microbe that modulates an activity of a target cell or virus, the method comprising: (a) forming a test sample comprising a plurality of target cells or viruses, and a mixed microbial population (e.g., a microbiome); (b) forming droplets from the test sample such that each droplet contains no more than one microbe from the mixed microbial population and a predetermined number of target cells or viruses; (c) incubating the droplets under conditions and for a period of time sufficient to detect a predetermined amount of activity of the target cells or viruses (e.g., cell replication and/or cell survival) in the absence of a microbe; (d) isolating a droplet in which the target cell or virus had a different amount of activity than the predetermined amount; and/or (e) sequencing DNA from the microbe in the isolated droplet to identify the microbe. In some embodiments, the droplet is isolated by FACS. In certain embodiments, the target cell or virus is fluorescently labeled prior to the step (d) of the methods provided herein. In some embodiments, the target cell or virus may express a fluorescent protein, or may be fluorescently labeled with a dye, a probe, or an antibody in a way such that the activity of the target cell or virus can be measured by a change in fluorescence.

In certain aspects, provided herein is a method for identifying a microbe that modulates an activity of a target cell or virus, the method comprising (a) forming a test sample comprising a plurality of target cells or viruses, and a mixed microbial population (e.g., a microbiome); (b) distributing the test sample into microwells such that each microwell contains no more than one microbe from the mixed microbial population and a predetermined number of target cells or viruses; (c) incubating the microwells under conditions and for a period of time sufficient to detect a predetermined amount of activity of the target cells or viruses (e.g., cell replication and/or cell survival) in the absence of a microbe; (d) detecting a microwell in which the target cell or virus had a different amount of activity from the predetermined amount; and/or (e) sequencing DNA from the microbe in the detected microwell to identify the microbe. In some embodiments, the microwell is detected by fluorescent microscopy. In certain embodiments, the target cell or virus is fluorescently labeled prior to the step (d) of the methods provided herein. In some embodiments, the target cell or virus may express a fluorescent protein, or may be fluorescently labeled with a dye, a probe, or an antibody in a way such that the activity of the target cell or virus can be measured by a change in fluorescence.

In certain aspects, provided herein is a method for identifying a microbe that inhibits replication or survival of a microbial cell, the method comprising: (a) forming a test sample comprising a plurality of microbial cells, a mixed microbial population (e.g., a microbiome), a set of barcoded primers comprising a universal priming sequence, a unique barcode and a universal microbial sequence, and a plurality of universal primers specific for the universal priming sequence; (b) forming droplets from the test sample such that each droplet contains no more than one microbe from the mixed microbial population, a predetermined number of microbial cells, no more than 1 barcoded primer, and a plurality of universal primers; (c) incubating the droplets under conditions and for a period of time sufficient to detect a predetermined amount of replication or survival of the microbial cells in the absence of a microbe; (d) performing an emulsion PCR reaction to generate barcode-labeled amplification product, wherein each unique barcode is associated with a single droplet in which the amplification took place; and/or (e) performing next-generation sequencing analysis of the amplification products to identify microbial sequences from droplets in which the microbial cell had a different amount of replication or survival from the predetermined amount.

In yet another aspect, provided herein is a method for identifying a microbe that inhibits replication or survival of a microbial cell, the method comprising: (a) forming a test sample comprising a plurality of microbial cells, a mixed microbial population (e.g., a microbiome), a set of barcoded primers comprising a universal priming sequence, a unique barcode and a universal microbial sequence, and a plurality of universal primers specific for the universal priming sequence; (b) distributing the test sample into microwells such that each microwell contains no more than one microbe from the mixed microbial population, a predetermined number of microbial cells, no more than 1 barcoded primer, and a plurality of universal primers; (c) incubating the microwells under conditions and for a period of time sufficient to detect a predetermined amount of replication or survival of the microbial cells in the absence of a microbe; (d) performing an emulsion PCR reaction to generate barcode-labeled amplification product, wherein each unique barcode is associated with a single microwell in which the amplification took place; and/or (e) performing next-generation sequencing analysis of the amplification products to identify microbial sequences from droplets in which the microbial cell had a different amount of replication or survival from the predetermined amount.

In certain aspects, provided herein is a method for identifying a microbe that modulates an enzymatic reaction of a target substrate, the method comprising: (a) forming a test sample comprising a plurality of target substrates and a mixed microbial population (e.g., a microbiome); (b) forming droplets from the test sample such that each droplet contains no more than one microbe from the mixed microbial population and a predetermined number of target substrates; (c) incubating the droplets under conditions and for a period of time sufficient to detect a predetermined amount of enzymatic reaction of the target substrate in the absence of a microbe; (d) isolating a droplet in which the target substrate underwent a different amount of enzymatic reaction from the predetermined amount; and/or (e) sequencing DNA from the microbe in the isolated droplet to identify the microbe. In some embodiments, the target substrate is fluorescently labeled prior to the step d of the method. For example, in some embodiments, the target substrate may be linked to a fluorescent protein, or may be fluorescently labeled with a dye, a probe, or an antibody in a way that the enzymatic reaction of the target substrate is measured by the level of the fluorescence. In some embodiments, the droplet is isolated by FACS.

In some aspects, provided herein is a method for identifying a microbe that modulates an enzymatic reaction of a target substrate, the method comprising: (a) forming a test sample comprising a plurality of target substrates and a mixed microbial population; (b) distributing the test sample into microwells such that each microwell contains no more than one microbe from the mixed microbial population and a predetermined number of target substrates; (c) incubating the microwells under conditions and for a period of time sufficient to detect a predetermined amount of enzymatic reaction of the target substrate in the absence of a microbe; (d) detecting a microwell in which the target substrate underwent a different amount of enzymatic reaction from the predetermined amount; and/or (e) sequencing DNA from the microbe in the detected microwell to identify the microbe. In some embodiments, the target substrate is fluorescently labeled prior to the step d of the method. For example, in some embodiments, the target substrate may be linked to a fluorescent protein, or may be fluorescently labeled with a dye, a probe, or an antibody in a way that the enzymatic reaction of the target substrate is measured by the level of the fluorescence. In some embodiments, the microwell is detected by fluorescent microscopy. As described herein, numerous embodiments are further provided that can be applied to any aspect of the present invention and/or combined with any other embodiment described herein.

In some aspects, provided herein is a method for identifying a microbe that protects a host cell against viral infection, the method comprising: (a) forming a test sample comprising a plurality of host cells, a mixed microbial population and a plurality of target viruses; (b) forming droplets from the test sample such that each droplet contains no more than one microbe from the mixed microbial population, a predetermined number of target viruses, and a predetermined number of host cells; (c) incubating the droplets under conditions and for a period of time sufficient to detect a predetermined amount of activity of the host cells in the presence of the target viruses but absence of a microbe; (d) isolating a droplet in which the host cell had a different amount of activity than the predetermined amount; and (e) sequencing DNA from the microbe in the isolated droplet to identify the microbe.

In some aspects, provided herein is a method for identifying a microbe that protects a host cell against viral infection, the method comprising: (a) forming a test sample comprising a plurality of host cells, a plurality of target viruses, and a mixed microbial population; (b) distributing the test sample into microwells such that each microwell contains no more than one microbe from the mixed microbial population, a predetermined number of target viruses, and a predetermined number of host cells; (c) incubating the microwells under conditions and for a period of time sufficient to detect a predetermined amount of activity of the host cells in the presence of the target viruses but absence of a microbe; (d) detecting a microwell in which the host cell had a different amount of activity from the predetermined amount; and (e) sequencing DNA from the microbe in the detected microwell to identify the microbe.

In some embodiments, the activity of the host cell is cell death. In some embodiments, the predetermined amount of cell death is at least a 2-fold reduction in cell number. In certain embodiments, the microbe inactivated or eliminated the target virus.

The mixed microbial population may be a microbiome of any multicellular organism. In certain embodiments of the methods provided herein, the mixed microbial population is the microbiome of a wild animal. The wild animal may be, for example, a mammal, a bird, a reptile, a fish, an amphibian, or an invertebrate. In certain embodiments of the methods provided herein, the mixed microbial population is a human microbiome, e.g., a human gut microbiome.

In certain embodiments of the methods provided herein, the target cell is a bacterial pathogen. In some embodiments, the bacterium is of the genus Aspergillus, Brugia, Candida, Chlamydia, Clostridium, Coccidia, Cryptococcus, Dirofilaria, Gonococcus, Enterococcus, Escherichia, Helicobacter, Histoplasma, Leishmania, Mycobacterium, Mycoplasma, Paramecium, Pertussis, Plasmodium, Mycobacterium, Mycoplasma, Pneumococcus, Pneumocystis, Pseudomonas, Rickettsia, Salmonella, Shigella, Staphylococcus, Streptococcus, Toxoplasma or Vibriocholerae. In certain embodiments, the bacterium is of the species Acinetobacter baumannii, Neisseria gonorrhea, Neisseria meningitidis, Mycobacterium tuberculosis, Candida albicans, Candida tropicalis, Trichomonas vaginalis, Haemophilus vaginalis, Group B Streptococcus sp., Microplasma hominis, Mycoplasma adleri, Dermatophilus congolensis, Diplorickettsia massiliensis, Mycoplasma agalactiae, Mycoplasma amphoriforme, Mycoplasma fermentans, Mycoplasma genitalium, Mycoplasma haemofelis, Mycoplasma hominis, Mycoplasma hyopneumoniae, Mycoplasma hyorhinis, Mycoplasma pneumoniae, Hemophilus ducreyi, Klebsiella pneumoniae, Granuloma inguinale, Lymphopathia venereum, Treponema pallidum, Mycobacterium tuberculosis, Brucella abortus. Brucella melitensis, Brucella suis, Brucella canis, Campylobacter fetus, Campylobacter fetus intestinalis, Leptospira pomona, Peptostreptococcus anaerobius, Peptostreptococcus asaccharolyticus, Listeria monocytogenes, Staphylococcus aureus, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Streptococcus pneumoniae, Streptococcus pyogenes, Ureaplasma gallorale, Corynebacterium pyogenes, Pasteuria ramosa, Actinobaccilus seminis, Mycoplasma bovigenitalium, Aspergillus fumigatus, Absidia ramosa, Trypanosoma equiperdum, Babesia caballi, Clostridium tetani or Clostridium botulinum.

In some embodiments of the methods provided herein, the target cell is a cancer cell. The cancer cell may be a primary cancer cell (i.e., obtained directly from a patient) or a cancer cell line. In some embodiments, the cancer cell may be from a cancer of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus.

In certain embodiments of the methods provided herein, the target cell is an immune cell. In certain embodiments, the immune cell may be a B cell, a T cell, a natural killer cell or a myeloid cell (e.g., a monocyte, a macrophage, an eosinophil, a mast cell, a basophil, or a granulocyte).

In some embodiments, the methods provided herein further comprises culturing the identified microbe (e.g., in a dish, in the droplet, in the microwell, etc.).

In some embodiments, the methods provided herein further comprise obtaining the mixed microbial population. In some embodiments, the mixed microbial population is obtained from a solid sample, a plant sample, a sea sample, a fecal sample, an oral sample, a saliva sample, a skin swab sample, etc.

### BRIEF DESCRIPTION OF THE DRAWINGS

FIG. 1 shows a schematic representation of an exemplary method provided herein. Microbiome sample (as a suspension in buffer) and fluorescently-labeled target cell (e.g., pathogenic bacteria, also as suspension in buffer or as a starter culture) are encapsulated in droplets such that each droplet statistically contains no more than one microorganism from the microbiome and one target pathogen. The droplets are incubated for an appropriate length of time (e.g., 30 min-3 h, or otherwise, depending on specific details of application), and then sorted by FACS. Droplets where the growth of the pathogen is not inhibited will appear with higher fluorescence, compared to droplets where microorganism inhibited pathogen growth. The latter droplets are sorted out and the contained microorganism from the microbiome is subjected to sequencing to identify microorganism and/or is cultured to amplify the microorganism.

FIG. 2 includes two panels. The left panel depicts droplets visualized by light microscopy (droplet diameter=30 um) containing a limiting dilution of microbes from a microbiome and fluorescently-labeled target bacteria, while the right panel shows same field in a fluorescence channel. A droplet with many target pathogens can be seen on the left side of the left panel, and a droplet with a single target pathogen near the middle of the left panel. The right panel shows that the latter droplet also contains a microorganism from the microbiome.

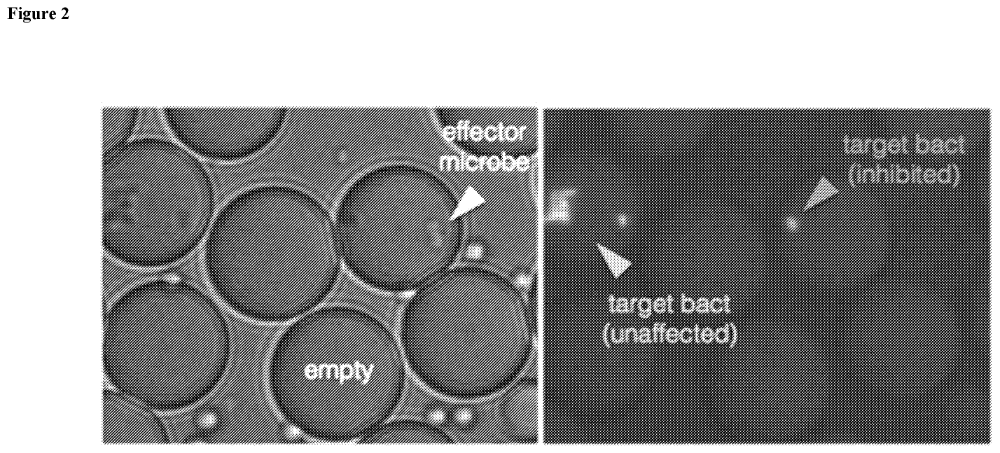

FIG. 3 shows a schematic representation of an exemplary method provided herein that is based on the identification of an effector microbe through sequencing without sorting. Microbiome sample (as a suspension in buffer) and a target cell (e.g., pathogenic bacteria, also as suspension in buffer or as a starter culture) are encapsulated in droplets into droplets along with a mixture of barcoded primers (recognizing universal microbial genes) such that each droplet statistically contains a single set of barcoded primers. Following incubation, an emulsion PCR is performed such that each template within the droplet is barcoded. Next generation sequencing (NGS) is next performed. The outcome is that barcodes tagging abundant templates represent ineffective microorganisms (i.e., microorganisms that cannot inhibit the growth of the target pathogen), whereas barcodes tagging rare templates represent effective microorganisms (i.e., microorganisms that inhibit the growth of the target pathogen). Identical barcodes on microorganism and target pathogen associate them to the same original droplet.

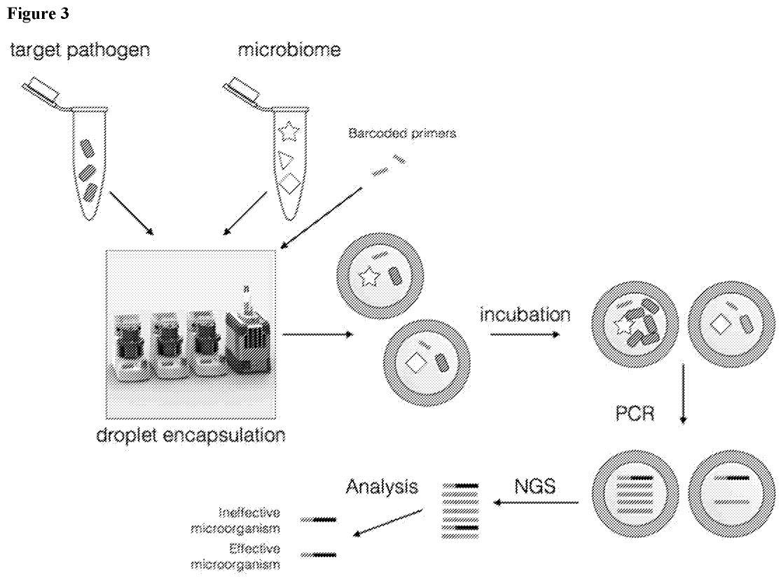

FIG. 4 shows a schematic representation of an exemplary method provided herein that is based on the identification of a microbe effective against a target virus. A microbiome sample (as a suspension in buffer), a plurality of target viruses, and a plurality of fluorescently-labeled host cells (also as suspension in buffer or as a starter culture) are encapsulated in droplets such that each droplet statistically contains no more than one microorganism from the microbiome, one target virus and one host cell. The droplets are incubated for an appropriate length of time (e.g., 30 min-3 h, or otherwise, depending on specific details of application), and then sorted by FACS. Droplets where the host cell is infected by the virus have a lower fluorescence (e.g., by losing the staining with the fluorescent cell viability dye), compared to droplets where microorganism inactivates the virus and thereby protecting the host cell against the viral infection. The latter droplets are sorted out and the contained microorganism from the microbiome is subjected to sequencing to identify the microorganism and/or is cultured to amplify the microorganism.

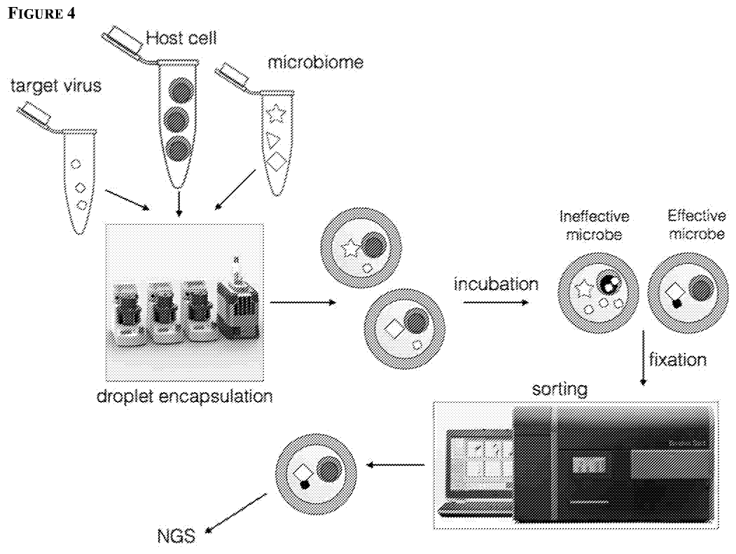

### DETAILED DESCRIPTION

### General

In certain aspects, the present disclosure describes methods and compositions for isolating microbes from mixed microbial populations that perform desirable functions, identifying such microbes, and/or using such microbes in industrial or clinical settings. In some embodiments, the methods may include distributing individual microbes from a microbiome into multiple droplets or microwells along with specific targets. The individual microbes are allowed to interact with their targets, following which the emulsion or microwells are analyzed by flow cytometry, microcopy, and/or next-generation sequencing (NGS), in order to identify strains that have the desired effect on the target (e.g. inhibit target cell growth, induce target cell killing, cleave target peptide substrate, etc.).

### Definitions

For convenience, certain terms employed in the specification, examples, and appended claims are collected here.

The articles “a” and “an” are used herein to refer to one or to more than one (e.g., to at least one) of the grammatical object of the article. By way of example, “an element” means one element or more than one element.

“About” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, the term “administering” means providing a pharmaceutical agent or composition to a subject, and includes, but is not limited to, administering by a medical professional and self-administering.

The term “barcoded primer” refers to a primer comprising a unique nucleotide sequence. The minimal length of this nucleotide sequence depends on the total number of primers that need to be uniquely labeled. For example, a nucleotide sequence that is 4 nucleotides long can have 256 different sequences, which can uniquely label up to 256 primers. The term “barcode-labeled amplification product is generated with these “barcoded primer” by PCR amplification reaction.

The term “decrease” or “deplete” means a change, such that the difference is, depending on circumstances, reduced by at least 10%, 20%, 30%, 40%, 50%, 60%, 70%, 80%, 90%, or more when subjected to a test condition (e.g., cultured in the presence of a test microbe from a microbiome) compared when not subjected to the test condition (e.g., when cultured under otherwise identical conditions but not in the presence of the test microbe from the microbiome).

The term “increase” means a change, such that the difference is, depending on circumstances, increased by at least 10%, 20%, 30%, 40%, 50%, 60%, 70%, 80%, 90%, 2-fold, 3-fold, 4-fold, 5-fold or more when subjected to a test condition (e.g., cultured in the presence of a test microbe from a microbiome) compared when not subjected to the test condition (e.g., when cultured under otherwise identical conditions but not in the presence of the test microbe from the microbiome).

The term “isolated” or “enriched” encompasses a microbe, bacteria or other entity or substance that has been (1) separated from at least some of the components with which it was associated when initially produced (whether in nature or in an experimental setting), and/or (2) produced, prepared, purified, and/or manufactured by the hand of man. Isolated microbes may be separated from at least about 10%, about 20%, about 30%, about 40%, about 50%, about 60%, about 70%, about 80%, about 90%, or more of the other components with which they were initially associated. In some embodiments, isolated microbes are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a microbe or other material that has been separated from at least some of the components with which it was associated either when initially produced or generated (e.g., whether in nature or in an experimental setting), or during any time after its initial production. A microbe or a microbial population may be considered purified if it is isolated at or after production, such as from a material or environment containing the microbe or microbial population, and a purified microbe or microbial population may contain other materials up to about 10%, about 20%, about 30%, about 40%, about 50%, about 60%, about 70%, about 80%, about 90%, or above about 90% and still be considered “isolated.” In some embodiments, purified microbes or microbial population are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components thereof are generally purified from residual habitat products.

The term “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity, or process. In certain instances, such regulation may be contingent on the occurrence of a specific event, such as activation of a signal transduction pathway, and/or may be manifest only in particular cell types.

“Microbiome” refers to the communities of microbes that live in or on a non-microbial organism (e.g., a plant, an animal, etc.), or in a certain environment (e.g., ocean, freshwater lakes, soil, etc.) both sustainably and transiently, including eukaryotes, archaea, bacteria, and viruses (including bacterial viruses (i.e., phage)).

A “cytotoxic” activity or bacterium includes the ability to kill a target cell, such as a pathogenic bacterial or fungal cell, cancer cell, pathogenic protozoa, etc. A “cytostatic” activity or bacterium includes the ability to inhibit, partially or fully, growth, metabolism, and/or proliferation of a target cell, such as a pathogenic bacterial or fungal cell, cancer cell, pathogenic protozoa, etc.

The terms “polynucleotide” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

Unless otherwise defined herein, scientific and technical terms used in this application shall have the meanings that are commonly understood by those of ordinary skill in the art. Generally, nomenclature and techniques relating to chemistry, molecular biology, cell and cancer biology, immunology, microbiology, pharmacology, and protein and nucleic acid chemistry, described herein, are those well-known and commonly used in the art.

### Microbiome Collection

In certain embodiments, the methods provided herein include the collection and/or analysis of a mixed microbial population. In certain embodiments, the mixed microbial population is a microbiome.

In some embodiments, the microbiome is obtained from an animal. In some embodiments, the microbiome is a human microbiome. In some embodiments, the microbiome is from a non-human animal. In some embodiments, the non-human animal is a domesticated and/or a laboratory animal. For example, in some embodiments the microbiome is from a mouse, rat, rabbit, pig, bovine (e.g., cow, bull, buffalo), deer, sheep, goat, llama, chicken, cat, dog, ferret, or a primate (e.g., marmoset, rhesus monkey). In some embodiments, the microbiome is from a mammal.

In some embodiments, the microbiome is from a wild animal. In certain embodiments, the wild animal may be a mammal, a bird, a reptile, a fish, an amphibian, or an invertebrate.

In certain embodiments, the microbiome is obtained from a fecal sample. In some embodiments, the fecal matter may be collected from more than three individual animals per species. In some embodiments, the fecal matter may be collected more than three times per individual animal. Fecal matter may be rapidly suspended in buffer (e.g. PBS) or culture medium (e.g., LB, 2YT, or a broad-range of growth media including eukaryotic cell growth medium such as DMEM, RPMI, etc.) and large particles or undigested material may be removed from suspension by centrifugation or filtration (e.g., filtering through >20 microns filter). In some embodiments, microbiome may be obtained from a soil sample, a plant sample, or a sea sample. In some embodiments, for purposes of maintaining a database, DNA and RNA may be extracted from a sample of this suspension using standard protocols and kits (e.g. Qiagen QIAmp® DNA Stool kits) and stored frozen. In some embodiments, for purposes of analyzing proteins, samples may be lysed by standard lysis buffers (e.g., Pierce B-PER or M-PER buffers) or using mechanical disruption methods.

In some embodiments, the microbiome sample is maintained alive (unfrozen and unfixed) for a period of more than 24 hours.

In some embodiments, the microbiome is from a plant (e.g., a microbiome associated with a plant root, leaf, stem, flower, fruit, etc.).

### Modulating Activity of a Target Cell

In certain embodiments, the methods provided herein include the step of culturing a microbe from a mixed microbial population with a target cell. In certain embodiments, the target cell can be any type of cell. For example, in some embodiments, the cell may be a prokaryotic or a eukaryotic cell. In some embodiments, the target cell may be pathogenic or non-pathogenic. In some embodiments, the target cell can be a bacterial pathogen, a fungus, a protest, a yeast cell or a mammalian cell. It may be a primary cell, such as a cell from a patient, or it can be an established or engineered cell line.

In some embodiments, the target cell is a bacterial pathogen. In some embodiments, the bacterium can be any pathogenic bacterium. In some embodiments, the bacterium is of the genus Aspergillus, Brugia, Candida, Chlamydia, Clostridium, Coccidia, Cryptococcus, Dirofilaria, Gonococcus, Enterococcus, Escherichia, Helicobacter, Histoplasma, Leishmania, Mycobacterium, Mycoplasma, Paramecium, Pertussis, Plasmodium, Mycobacterium, Mycoplasma, Pneumococcus, Pneumocystis, Pseudomonas, Rickettsia, Salmonella, Shigella, Staphylococcus, Streptococcus, Toxoplasma or Vibriocholerae. In certain embodiments, the bacterium is of the species Acinetobacter baumannii, Neisseria gonorrhea, Neisseria meningitidis, Mycobacterium tuberculosis, Candida albicans, Candida tropicalis, Trichomonas vaginalis, Haemophilus vaginalis, Group B Streptococcus sp., Microplasma hominis, Mycoplasma adleri, Dermatophilus congolensis, Diplorickettsia massiliensis, Mycoplasma agalactiae, Mycoplasma amphoriforme, Mycoplasma fermentans, Mycoplasma genitalium, Mycoplasma haemofelis, Mycoplasma hominis, Mycoplasma hyopneumoniae, Mycoplasma hyorhinis, Mycoplasma pneumoniae, Hemophilus ducreyi, Klebsiella pneumoniae, Granuloma inguinale, Lymphopathia venereum, Treponema pallidum, Mycobacterium tuberculosis, Brucella abortus. Brucella melitensis, Brucella suis, Brucella canis, Campylobacter fetus, Campylobacter fetus intestinalis, Leptospira pomona, Peptostreptococcus anaerobius, Peptostreptococcus asaccharolyticus, Listeria monocytogenes, Staphylococcus aureus, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Streptococcus pneumoniae, Streptococcus pyogenes, Ureaplasma gallorale, Corynebacterium pyogenes, Pasteuria ramosa, Actinobaccilus seminis, Mycoplasma bovigenitalium, Aspergillus fumigatus, Absidia ramosa, Trypanosoma equiperdum, Babesia caballi, Clostridium tetani or Clostridium botulinum.

In some embodiments, the target cell is a cancer cell. The terms “cancer” or “tumor” refer to the presence of cells possessing characteristics typical of cancer-causing cells, such as uncontrolled proliferation, immortality, metastatic potential, rapid growth and proliferation rate, and certain characteristic morphological features. Cancer is generally associated with uncontrolled cell growth, invasion of such cells to adjacent tissues, and the spread of such cells to other organs of the body by vascular and lymphatic menas. Cancer invasion occurs when cancer cells intrude on and cross the normal boundaries of adjacent tissue, which can be measured by assaying cancer cell migration, enzymatic destruction of basement membranes by cancer cells, and the like. In some embodiments, a particular stage of cancer is relevant and such stages can include the time period before and/or after angiogenesis, cellular invasion, and/or metastasis. Cancer cells are often in the form of a solid tumor, but such cells may exist alone within an animal, or may be a non-tumorigenic cancer cell, such as a leukemia cell.

In some embodiments, the cell can be from any type of cancer, including, but not limited to, cancer cell from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the target cell is an immune cell. As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

In some aspects, the methods provided herein relate to methods for identifying a microbe that modulates an activity of the target cell. The activity of a target cell could be any activity of that cell, including but is not limited to cell replication, cell survival, or cell activation. In some embodiments, the method relates to identifying a microbe that inhibits or reduces the cell replication or cell survival of the target cell. In some embodiments, the method relates to the identification of a microbe that inhibits and/or reduces the production of a product produced by the target cell (e.g., production of a bacterial toxin from a bacterial pathogen, production of a cytokine from an immune cell, production of an immune checkpoint protein from a cancer cell). For example, in some embodiments, the method relates to the identification of a microbe that modulates the level of expression or activity of a biomarker that has been determined to be predictive of cancer and/or immune therapy.

In some embodiments, the target cell is an immune cell, and the method relates to the identification of a microbe that modulates a particular immune response of the immune cell. As used herein, the term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages. In some other embodiments, the method could also be for identifying a microbe that modulates the phagocytic activity of an immune cell such as monocyte, or macrophage.

In some embodiments the target cell is a vertebrate cell, such as a mammalian cell including non-primate cells (e.g., cells from a cow, pig, horse, donkey, goat, camel, cat, dog, guinea pig, rat, mouse, sheep) and primate cells (e.g., a cell from a monkey, gorilla, chimpanzee) In some embodiments the cell is a primary cell. In some embodiments, the cell is a cell line. Examples of cell lines include, but are not limited to, P19 cells, HUVAC cells, 293-T cells, 3T3 cells, 721 cells, 9L cells, A2780 cells, A172 cells, A253 cells, A431 cells, CHO cells, COS-7 cells, HCA2 cells, HeLa cells, Jurkat cells, NIH-3T3 cells and Vero cells.

In some embodiments, the target cell is detectably labeled. Examples of detectable labels include, but are not limited to, fluorescent moieties, radioactive moieties, paramagnetic moieties, luminescent moieties and/or colorometric moieties. In some embodiments, the target cell is genetically modified to express the detectable moiety (e.g. the target cell is modified to express a fluorescent protein, such as GFP). In some embodiments, the target cell is directly labeled with the detectable moiety (e.g., a fluorescent dye). In some embodiments, the target cell is indirectly labeled (e.g., it is bound by an antibody or other protein that is directly or indirectly labeled with a detectable moiety).

In some embodiments the target cell is labeled with a fluorescent moiety. Examples of fluorescent moieties include, but are not limited to, Allophycocyanin, Fluorescein, Phycoerythrin, Peridinin-chlorophyll protein complex, Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 633, Alexa Fluor 635, Alexa Fluor 647, Alexa Fluor 660, Alexa Fluor 680, Alexa Fluor 700, Alexa Fluor 750, Alexa Fluor 790, EGFP, mPlum, mCherry, mOrange, mKO, EYFP, mCitrine, Venus, YPet, Emerald, Cerulean and CyPet.

In some embodiments, the target is a virus. In some embodiments, the target can be any virus. In some embodiments, the virus is Human Papilloma Virus (HPV), HBV, hepatitis C Virus (HCV), human immunodeficiency virus (HIV-1, HIV-2), varicella virus, herpes virus, Epstein Barr Virus (EBV), mumps virus, rubella virus, rabies virus, measles virus, viral hepatitis, viral meningitis, cytomegalovirus (CMV), HSV-1, HSV-2, or influenza virus.

In certain embodiments, the methods provided herein include the step of culturing a microbe from a mixed microbial population with a host cell and a target virus. In certain embodiments, the host cell can be any type of cell. For example, in some embodiments, the host cell may be a prokaryotic or a eukaryotic cell. In some embodiments, the host cell may be pathogenic or non-pathogenic. In some embodiments, the host cell can be a bacterial pathogen, a fungus, a protest, a yeast cell or a mammalian cell. It may be a primary cell, such as a cell from a patient, or it can be an established or engineered cell line. The target virus may be any pathogenic virus described herein, including but not limited to, Human Papilloma Virus (HPV), HBV, hepatitis C Virus (HCV), human immunodeficiency virus (HIV-1, HIV-2), varicella virus, herpes virus, Epstein Barr Virus (EBV), mumps virus, rubella virus, rabies virus, measles virus, viral hepatitis, viral meningitis, cytomegalovirus (CMV), HSV-1, HSV-2, or influenza virus. In some embodiments, the microbe protect the host cell from viral infection by inactivating or eliminating the virus. The term “inactivating a virus” refers to any process that renders a virus inactive, or unable to infect, such as, chemically altering the lipid or protein coats of the virus, denaturing the virus, etc.

### Modulating Enzymatic Reaction of a Target Substrate

In some aspects, the methods provided herein relate to methods for identifying a microbe that modulates an enzymatic reaction of a target substrate. In certain embodiments, the target substrate may be protein, peptide, DNA, RNA, or small molecule compound. The target substrate could be isolated directly from a patient, an organism, or a cell or can be chemically synthesized. In some embodiments, the target protein is a recombinant protein, peptide, DNA, or RNA which is made or synthesized in vitro using well-known standard methods.

In certain embodiments, the enzymatic reaction could be a reaction of any kind. Exemplary enzymatic reasons include but are not limited to cleavage of a protein, peptide, DNA or RNA molecule, protein aggregation, phosphorylation or de-phosphorylation of a protein or peptide, replication of DNA, transcription or translation of mRNA, or chemical interconversions in the metabolic or biosynthetic pathways. In some embodiments, the test sample comprises a plurality of target substrates, a mixed microbial population, as well as other components that are required for the enzymatic reaction to occur.

In some embodiments, the enzymatic reaction of the target substrate is detectable and/or quantifiable. In some embodiments, the target substrate is labeled such that the enzymatic reaction results in the modulation of a detectable signal. For example, in some embodiments, the enzymatic reaction results in the cleavage of a protein, peptide or nucleic acid which results in the separation of a signaling fluorophore from a quenching moiety.

Mixing Microbe with Target Cell or Target Substrate

In some aspects, methods of provided herein include steps of forming a test sample comprising a plurality of targets (e.g., target cells or substrates) and a mixed microbial population, and generating droplets from the test sample or distributing the test sample into microwells so that each droplet or microwell contains no more than one microbe from the mixed microbial population and a predetermined number of target cells or target substrates.

### Droplet Generation

In some embodiments, the method includes preparing droplets from the test sample such that the microbes from the mixed microbial population are encapsulated in droplets. In some embodiments, the droplets have a diameter of about 5, 10, 15, 20, 25, 30, 35, 40, 45, 50, 55, 60, 65, 70, 75, 80, 85, 90, 95 or 100 microns. In some embodiments, the droplets are 30-micron diameter droplets. In some embodiments, the droplets are double-encapsulated. In some embodiments, the droplets are water-in-oil emulsions. In some embodiments, the droplets are semi-solid. For example, the droplets may be gel, hydrogel, or sol-gel which is made from agarose, alginate, gelatin, extracellular matrix proteins (e.g., collagen), etc. In some embodiments, the droplets (e.g., those made of low-melting agarose) are solidified by decreasing temperature. In some embodiments, the droplets are solidified by cross-linking (e.g., by calcium crosslinking of alginate, transglutaminase crosslinking of gelatin, or UV crosslinking of other matrices).

The droplet forming process can be performed using any appropriate method known in the art. For example, in some embodiments a Dolomite Microencapsulator-1 system may be used to form, e.g., 30-micron diameter, double-encapsulated droplets. In some embodiments, a OneTouch instrument (ion Torrent) may be used.

In some embodiments, at least 10, 50, 100, 250, 500, 750, 1,000, or more droplets per microbe are made. For example, in some embodiments, at least 1,000, 2,500, 5,000, 7,500, 10,000, 25,000, 50,000, 75,000, 100,000, 250,000, 500,000, 750,000, or 1,000,000, droplets per microbe may be made. In some embodiments, 1,000,000 droplets per microbiome may be made. Droplets may be made with the following inputs:
Input A: microbiome suspension; and
Input B: target cell/virus suspension or target substrate.

If the droplets are for pathogenic virus-inactivating microbe detection, the droplets may be made with the following inputs:
Input A: microbiome suspension;
Input B: target virus; and
Input C: host cell.

The target cell or target substrate could be fluorescently-labeled as described herein. Each droplet may contain statistically no more than 1 entity from input A, and more than 1 entity from input B, depending on the application. In some embodiments, the predetermined number of target cells in a droplet is no more than one target cell (e.g., zero or one target cell). The inner droplet environment may comprise the mixture of the solutions of inputs A and B. In some embodiments, each droplet may include no more than one cell from input A, virus particles at a specific multiplicity of infection (i.e., virus-to-host cell ratio) from input B, and more than one cell of input C (depending on said ratio).

### Reaction Conditions

In certain embodiments, the methods of the present disclosure include a step of incubating the droplets or microwells containing the test solution under conditions and for a period of time. In certain embodiments, the conditions and period of time are conditions and a period of time sufficient to detect a desired activity, if present, of the microbes from the mixed microbial population on the test cell or test substrate. For example, in certain embodiments, the desired activity is inhibition of replication of the test cells. In such embodiments, the test cells are cultured under conditions compatible with their division and for a period of time sufficient for the test cells to divide at least 1-time, 2-times, 3-times, 4-times, 5-times or more in the absence of the microbe from the mixed microbial population. In certain embodiments, the desired activity is the killing of the test cells. In such embodiments, the test cells are cultured under conditions and for a period of time under which they will survive in the absence of the microbe from the mixed microbial population.

The conditions may include appropriate culturing conditions for the test cell (e.g., temperature, humidity, concentration of CO₂, basal medium, serum, antibiotics, pH, reducing agent, or stimulus), or appropriate reaction conditions for the test substrate (e.g., temperature, enzyme concentration, pH, ionic strength, cofactors, or concentrations of substrate or enzymes).

In certain embodiments, the period of time may range from a few minutes to a few weeks depending on the application. For example, in some aspects, the methods of the present disclosure is for identifying a microbe that modulates the cell replication of the target cell. In some embodiment, the predetermined amount of the replication may be at least a 2-fold expansion. The conditions may be appropriate culturing conditions of the target cells, and the period of time may need to be longer than the doubling time of the target cell under this particular culture condition.

### Identification of Microbes

### Labeling

Certain embodiments of the methods provided herein include the use of a detectably labeled test cell or test substrate. Examples of detectable labels include, but are not limited to, fluorescent moieties, radioactive moieties, paramagnetic moieties, luminescent moieties and/or colorometric moieties.

In some embodiments, the methods provided herein include the use of a target cell that is fluorescently labeled. In some embodiments, the target cell is modified to express a fluorescent protein, such as GFP, RFP, or YFP, or a protein fused with such a fluorescent protein. For example, the genome of the target cell may be engineered to express a fluorescent protein or a protein fused with a fluorescent protein, or the target cell may be transiently transfected with an expression vector, which drives the expression of a fluorescent protein, or a protein fused with a fluorescent protein. Such expression of a fluorescent protein or a protein fused with a fluorescent protein could be constitutive, inducible, or tightly controlled by specific promoters and/or regulatory elements in the genome or the expression vector.

In some embodiments, the target cell may be fluorescently labeled with a dye, a probe, or an antibody in a way that the activity of the target cell is measured by the level of the fluorescence. The term “probe” refers to any molecule which is capable of selectively binding to a specifically intended target molecule, for example, a nucleotide transcript or protein encoded by or corresponding to a marker. Probes can be either synthesized by one skilled in the art, or derived from appropriate biological preparations. For purposes of detection of the target molecule, probes may be specifically designed to be labeled with a fluorescent protein or a fluorophore, as described herein. Examples of molecules that can be utilized as probes include, but are not limited to, RNA, DNA, proteins, antibodies, and organic molecules. These fluorescent dyes or probes could be cell impermeable or permeable. They could bind to specific components on the cell membrane, or to specific intracellular components. In some embodiments, an antibody may be used to label the target cell by binding to specific antigens located either on the cell membrane or inside the cell. In certain embodiments the probe is linked to a fluorescent moiety. Examples of fluorescent moieties include, but are not limited to, Allophycocyanin, Fluorescein, Phycoerythrin, Peridinin-chlorophyll protein complex, Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 633, Alexa Fluor 635, Alexa Fluor 647, Alexa Fluor 660, Alexa Fluor 680, Alexa Fluor 700, Alexa Fluor 750, Alexa Fluor 790, EGFP, mPlum, mCherry, mOrange, mKO, EYFP, mCitrine, Venus, YPet, Emerald, Cerulean and CyPet.

The way in which a target cell is labeled depends on the specific cellular activity sought to be detected. For example, a dye that labels a living cell and can be passed to daughter cells with several passages may be used to detect the cell proliferation. An antibody binds to a specific antigen may be used to detect the expression or secretion of the antigen by the target cell. In some embodiments, a target cell may be labeled by incubating the cell with fluorescently-labeled cargos (e.g., fluorescent beads or fluorescently-labeled cells) which can be uptaken by the target cell under appropriate conditions.

In some embodiments, the target substrate may be fluorescently labeled. The target substrate may be linked to a fluorescent moiety, GFP, RFP, YFP, Allophycocyanin, Fluorescein, Phycoerythrin, Peridinin-chlorophyll protein complex, Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 633, Alexa Fluor 635, Alexa Fluor 647, Alexa Fluor 660, Alexa Fluor 680, Alexa Fluor 700, Alexa Fluor 750, Alexa Fluor 790, EGFP, mPlum, mCherry, mOrange, mKO, EYFP, mCitrine, Venus, YPet, Emerald, Cerulean and CyPet. For example, the target substrate could be a target protein, peptide or nucleic acid that linked to a fluorescent moiety. In some embodiments, the target substrate may be fluorescently labeled with a dye, a probe, or an antibody in a way that the enzymatic reaction of the target substrate results in a change in the level of the fluorescence produced (e.g., by separating a fluorophore from a quencher or by bringing a fluorophore into proximity with a quencher). In some embodiment, the target substrate (e.g., protein, peptide, DNA, RNA or small molecule) may be covalently linked to a fluorophore. In some embodiment, the target substrate may be specifically bound by a probe (e.g., protein, peptide, nucleic acid, or antibody) that is labeled with a fluorophore or a fluorescent protein. The way in which a target cell is labeled depends on the enzymatic reaction sought to be detected. For example, cleavage of a protein or peptide can be detected by using fluorescently-labeled antibody that selectively binds to the cleaved product but not the full-length protein or peptide.

In some embodiments, the fluorescent labeling may be occur at any step prior to the detection of the activity of the target cell. For example, the target cell may be labeled before forming a test sample comprising a plurality of target cell and a mixed microbial population. The target cell may be labeled after forming the test sample, but before forming droplets from the test sample or distributing the test sample into microwells. The target cell may be labeled after forming droplets from the test sample or distributing the test sample into microwells, but before incubating the droplets or microwells under conditions and for a period of time sufficient to detect a predetermined amount of activity of the target cell. The target cell may be labeled after this incubation period, but before detection of the activity of the target cell.

In some embodiments, the fluorescent labeling may be occur at any step prior to the detection of the amount of enzymatic reaction the target substrate undergoes. For example, the target substrate may be labeled before forming a test sample comprising a plurality of target substrates and a mixed microbial population. The target substrate may be labeled after forming the test sample, but before forming droplets from the test sample or distributing the test sample into microwells. The target substrate may be labeled after forming droplets from the test sample or distributing the test sample into microwells, but before incubating the droplets or microwells under conditions and for a period of time sufficient to detect a predetermined amount of enzymatic reaction of the target substrate. The target substrate may be labeled after this incubation period, but before detection of the amount of enzymatic reaction the target substrate undergoes.

### Detection and Identification

In certain embodiments, following an incubation period at a specific temperature, the droplets or microwells are screened either by sorting them by FACS based on fluorescence, by light microscopy or fluorescence microscopy and image analysis, or by an in-droplet (emulsion) PCR followed by next-generation sequencing (NGS).

In some embodiments, the target cell or the target substrate is fluorescently labeled in a droplet before detection. Such droplets can be sorted by fluorescence activated cell sorting (FACS) based on fluorescence. The droplet with a desired level of florescence, which indicates the microbe in that droplet has the desired effect on the target cell, is collected. In some aspects, the droplet in which the target cell had a different amount (e.g., more or less) of activity than the predetermined amount may be isolated. In some aspects, the droplet in which the target substrate underwent a different amount (e.g., more or less) of enzymatic reaction from the predetermined amount may be isolated. For example, in some embodiments, the droplets which have fluorescence level of top 50% to top 0.1% (e.g., top 50%, top 45%, top 40%, top 35%, top 30%, top 25%, top 20%, top 10%, top 5%, top 1%, top 0.5%, top 0.2%, top 0.1%) of the full range of fluorescence of all droplets may be collected for downstream analysis. In some embodiment, the droplets which have fluorescence level of bottom 50% to bottom 0.1% (e.g., bottom 50%, bottom 45%, bottom 40%, bottom 35%, bottom 30%, bottom 25%, bottom 20%, bottom 10%, bottom 5%, bottom 1%, bottom 0.5%, bottom 0.2%, bottom 0.1%) of the full range of fluorescence of all droplets may be collected for downstream analysis. In some embodiments, the droplets may be collected as long as they are with positive fluorescence despite the level of the fluorescence. The collected droplets may then be sequenced with next-generation sequencing (NGS) technology to identify the microbes contained in these droplets.

In some embodiments, the target cell or the target substrate is fluorescently labeled in a microwell of a grid before detection. Such grid can be screened by fluorescence microscopy and image analysis based on fluorescence. The microwell with a desired level and/or pattern of florescence, which indicates the microbe in that microwell has the desired effect on the target cell, is identified, and the test sample in that microwell is collected. In some aspects, the microwell in which the target cell had a different amount (e.g., more or less) of activity than the predetermined amount may be isolated. In some aspects, the microwell in which the target substrate underwent a different amount (e.g., more or less) of enzymatic reaction from the predetermined amount may be isolated. For example, in some embodiments, the microwells which have fluorescence level of top 50% to top 0.1% (e.g., top 50%, top 45%, top 40%, top 35%, top 30%, top 25%, top 20%, top 10%, top 5%, top 1%, top 0.5%, top 0.2%, top 0.1%) of the full range of fluorescence of all microwells may be identified, and the test samples from these microwells are collected for downstream analysis. In some embodiment, the microwells which have fluorescence level of bottom 50% to bottom 0.1% (e.g., bottom 50%, bottom 45%, bottom 40%, bottom 35%, bottom 30%, bottom 25%, bottom 20%, bottom 10%, bottom 5%, bottom 1%, bottom 0.5%, bottom 0.2%, bottom 0.1%) of the full range of fluorescence of all microwells may be identified, and the test samples from these microwells are collected for downstream analysis. In some embodiments, the test samples may be collected from any microwells the have positive fluorescence despite the level of the fluorescence. The collected test samples may then be sequenced with next-generation sequencing (NGS) technology to identify the microbes contained in the identified microwells.

In some aspects, methods for identifying a microbe that inhibits replication or survival of a microbial cell is provided. The method comprises a step to form a test sample comprising a plurality of microbial cells, a mixed microbial population, a set of barcoded primers comprising a universal priming sequence, a unique barcode and a universal microbial sequence, and a plurality of universal primers specific for the universal priming sequence. The test sample then forms droplets or gets distributed into microwells such as each droplet or microwell contains no more than one microbe from the mixed microbial population, a predetermined number of microbial cells, no more than 1 barcoded primer, and a plurality of universal primers. After incubating such droplets or microwells under conditions and for a period of time sufficient to detect a predetermined amount of replication or survival of the microbial cells in the absence of a microbe, an emulsion PCR reaction is performed and barcode-labeled amplification products are generated. Each unique barcode is associated with a single droplet or microwell in which the amplification took place. In these methods, the target cell (i.e., the microbial cell) is not fluorescently labeled, but the microbe that inhibits replication or survival of the target cell (i.e., the microbial cell) may be identified performing next-generation sequencing analysis of the amplification products to identify microbial sequences from droplets or microwells in which the microbial cell had a different amount of replication or survival from the predetermined amount.

In some embodiments, a PCR reaction is carried out that fuses a specific viral template with an identifying template from the microbe, such that the amplicon contains a single copy of the microbial ID template and a number of viral templates corresponding to the number of viral particles. The products of this reaction are then analyzed by NGS.

### Validation

The methods of the present invention may further comprise culturing the identified microbe, and/or store the identified microbe under appropriate condition as frozen stocks. The function of the identified microbe may also be further validated by mixing a homogenous culture of the microbe with the target cells or substrate without forming droplets or distributing to microwells, and detecting the effect of the microbe on the target cell or substrate using the same or different labeling and/or detecting methods used in the original screening.

### EXAMPLES

The invention now being generally described, it will be more readily understood by reference to the following examples, which are included merely for purposes of illustration of certain aspects and embodiments of the present invention, and are not intended to limit the invention. As such, it will be readily apparent that any of the disclosed beneficial substances and therapies can be substituted within the scope of the present disclosure.

### Example 1

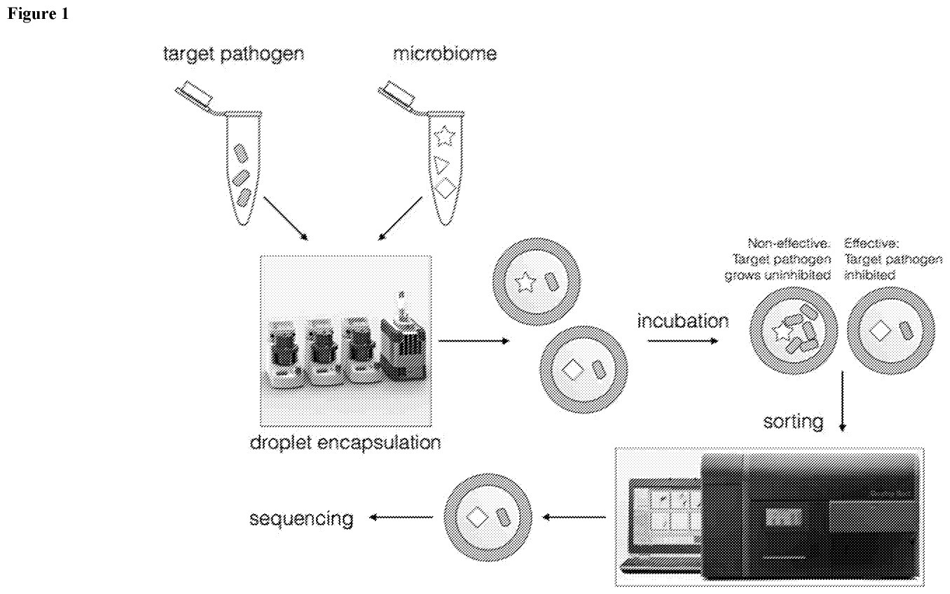

As illustrated in FIG. 1, microbes from gut microbiome samples were isolated and each individual was co-encapsulated with an individual target bacteria in droplets. Target bacteria expressed fluorescent proteins for detection and quantification of cells. After a 2-hour incubation, droplets were screened and sorted for growth inhibition of target bacteria, ones containing a single fluorescent bacteria were collected and sent to sequencing for effector microbes identification.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method for identifying a microbe that modulates an activity of a target cell or virus, the method comprising:
(a) forming a test sample comprising a plurality of target cells or viruses, and a mixed microbial population;
(b) forming droplets from the test sample such that each droplet contains no more than one microbe from the mixed microbial population and a predetermined number of target cells or viruses;
(c) incubating the droplets under conditions and for a period of time sufficient to detect a predetermined amount of activity of the target cells or viruses in the absence of a microbe;
(d) isolating a droplet in which the target cell or virus had a different amount of activity than the predetermined amount; and
(e) sequencing DNA from the microbe in the isolated droplet to identify the microbe.

2. The method of claim 1, wherein the activity of the target cell is cell replication or survival.

3. The method of claim 2, wherein the predetermined amount of cell replication is at least a 2-fold expansion.

4. The method of claim 1, wherein the mixed microbial population is a microbiome.

5. The method of claim 4, wherein the microbiome is the microbiome of a wild animal.

6. The method of claim 5, wherein the wild animal is a mammal, a bird, a reptile, a fish, an amphibian, or an invertebrate.

7. The method of any one of claims 1 to 6, wherein the target cell is a bacterial pathogen.

8. The method of claim 7, wherein the bacterial pathogen is Acinetobacter baumannii, Neisseria gonorrhea, Neisseria meningitidis, Mycobacterium tuberculosis, Candida albicans, Candida tropicalis, Trichomonas vaginalis, Haemophilus vaginalis, Group B Streptococcus sp., Microplasma hominis, Mycoplasma adleri, Dermatophilus congolensis, Diplorickettsia massiliensis, Mycoplasma agalactiae, Mycoplasma amphoriforme, Mycoplasma fermentans, Mycoplasma genitalium, Mycoplasma haemofelis, Mycoplasma hominis, Mycoplasma hyopneumoniae, Mycoplasma hyorhinis, Mycoplasma pneumoniae, Hemophilus ducreyi, Klebsiella pneumoniae, Granuloma inguinale, Lymphopathia venereum, Treponema pallidum, Mycobacterium tuberculosis, Brucella abortus. Brucella melitensis, Brucella suis, Brucella canis, Campylobacter fetus, Campylobacter fetus intestinalis, Leptospira pomona, Peptostreptococcus anaerobius, Peptostreptococcus asaccharolyticus, Listeria monocytogenes, Staphylococcus aureus, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Streptococcus pneumoniae, Streptococcus pyogenes, Ureaplasma gallorale, Corynebacterium pyogenes, Pasteuria ramosa, Actinobaccilus seminis, Mycoplasma bovigenitalium, Aspergillus fumigatus, Absidia ramosa, Trypanosoma equiperdum, Babesia caballi, Clostridium tetani or Clostridium botulinum.

9. The method of any one of claims 1 to 6, wherein the target cell is a cancer cell.

10. The method of claim 9, wherein the cancer cell is from a cancer of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus.

11. The method of any one of claims 1 to 6, wherein the target cell is an immune cell.

12. The method of claim 11, wherein the immune cell is a B cell, a T cell, a natural killer cell, or a myeloid cell.

13. The method of any one of claims 1 to 12, wherein the target cell or virus is fluorescently labeled prior to the step (d) of claim 1.

14. The method of any one of claims 1 to 13, wherein the target cell or virus expresses a fluorescent protein.

15. The method of claim 14, wherein the target cell or virus is fluorescently labeled with a dye, a probe, or an antibody in a way that the activity of the target cell or virus is measured by the level of the fluorescence.

16. The method of any one of claims 1 to 15, wherein the predetermined number of target cells is no more than one target cell, or the predetermined number of target viruses is no more than one target virus.

17. The method of any one of claims 1 to 16, wherein the droplet is isolated by FACS.

18. A method for identifying a microbe that inhibits replication or survival of a microbial cell, the method comprising:
(a) forming a test sample comprising a plurality of microbial cells, a mixed microbial population, a set of barcoded primers comprising a universal priming sequence, a unique barcode and a universal microbial sequence, and a plurality of universal primers specific for the universal priming sequence;
(b) forming droplets from the test sample such that each droplet contains no more than one microbe from the mixed microbial population, a predetermined number of microbial cells, no more than 1 barcoded primer, and a plurality of universal primers;
(c) incubating the droplets under conditions and for a period of time sufficient to detect a predetermined amount of replication or survival of the microbial cells in the absence of a microbe;
(d) performing an emulsion PCR reaction to generate barcode-labeled amplification product, wherein each unique barcode is associated with a single droplet in which the amplification took place;
(e) performing next-generation sequencing analysis of the amplification products to identify microbial sequences from droplets in which the microbial cell had a different amount of replication or survival from the predetermined amount.

19. The method of claim 18, wherein the mixed microbial population is a microbiome.

20. The method of claim 19, wherein the microbiome is the microbiome of a wild animal.

21. The method of claim 20, wherein the wild animal is a mammal, a bird, a reptile, a fish, an amphibian, or an invertebrate.

22. The method of any one of claims 18 to 21, wherein the microbial cell is a bacterial pathogen.

23. The method of claim 22, wherein the bacterial pathogen is Acinetobacter baumannii, Neisseria gonorrhea, Neisseria meningitidis, Mycobacterium tuberculosis, Candida albicans, Candida tropicalis, Trichomonas vaginalis, Haemophilus vaginalis, Group B Streptococcus sp., Microplasma hominis, Mycoplasma adleri, Dermatophilus congolensis, Diplorickettsia massiliensis, Mycoplasma agalactiae, Mycoplasma amphoriforme, Mycoplasma fermentans, Mycoplasma genitalium, Mycoplasma haemofelis, Mycoplasma hominis, Mycoplasma hyopneumoniae, Mycoplasma hyorhinis, Mycoplasma pneumoniae, Hemophilus ducreyi, Klebsiella pneumoniae, Granuloma inguinale, Lymphopathia venereum, Treponema pallidum, Mycobacterium tuberculosis, Brucella abortus. Brucella melitensis, Brucella suis, Brucella canis, Campylobacter fetus, Campylobacter fetus intestinalis, Leptospira pomona, Peptostreptococcus anaerobius, Peptostreptococcus asaccharolyticus, Listeria monocytogenes, Staphylococcus aureus, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Streptococcus pneumoniae, Streptococcus pyogenes, Ureaplasma gallorale, Corynebacterium pyogenes, Pasteuria ramosa, Actinobaccilus seminis, Mycoplasma bovigenitalium, Aspergillus fumigatus, Absidia ramosa, Trypanosoma equiperdum, Babesia caballi, Clostridium tetani or Clostridium botulinum.

24. The method of any one of claims 18 to 23, wherein the predetermined number of microbial cells is no more than one microbial cell.

25. The method of any one of claims 18 to 24, wherein the predetermined amount of replication is at least a 2-fold expansion.

26. A method for identifying a microbe that modulates an activity of a target cell or virus, the method comprising:
(a) forming a test sample comprising a plurality of target cells or viruses, and a mixed microbial population;
(b) distributing the test sample into microwells such that each microwell contains no more than one microbe from the mixed microbial population and a predetermined number of target cells or viruses;
(c) incubating the microwells under conditions and for a period of time sufficient to detect a predetermined amount of activity of the target cells or viruses in the absence of a microbe;
(d) detecting a microwell in which the target cell or virus had a different amount of activity from the predetermined amount; and
(e) sequencing DNA from the microbe in the detected microwell to identify the microbe.

27. The method of claim 26, wherein the activity of the target cell is cell replication or survival.

28. The method claim 27, wherein the predetermined amount of cell replication is at least a 2-fold expansion.

29. The method of claim 26, wherein the mixed microbial population is a microbiome.

30. The method of claim 29, wherein the microbiome is the microbiome of a wild animal.

31. The method of claim 30, wherein the wild animal is a mammal, a bird, a reptile, a fish, an amphibian, or an invertebrate.

32. The method of any one of claims 26 to 31, wherein the target cell is a bacterial pathogen.

33. The method of claim 32, wherein the bacterial pathogen is Acinetobacter baumannii, Neisseria gonorrhea, Neisseria meningitidis, Mycobacterium tuberculosis, Candida albicans, Candida tropicalis, Trichomonas vaginalis, Haemophilus vaginalis, Group B Streptococcus sp., Microplasma hominis, Mycoplasma adleri, Dermatophilus congolensis, Diplorickettsia massiliensis, Mycoplasma agalactiae, Mycoplasma amphoriforme, Mycoplasma fermentans, Mycoplasma genitalium, Mycoplasma haemofelis, Mycoplasma hominis, Mycoplasma hyopneumoniae, Mycoplasma hyorhinis, Mycoplasma pneumoniae, Hemophilus ducreyi, Klebsiella pneumoniae, Granuloma inguinale, Lymphopathia venereum, Treponema pallidum, Mycobacterium tuberculosis, Brucella abortus. Brucella melitensis, Brucella suis, Brucella canis, Campylobacter fetus, Campylobacter fetus intestinalis, Leptospira pomona, Peptostreptococcus anaerobius, Peptostreptococcus asaccharolyticus, Listeria monocytogenes, Staphylococcus aureus, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Streptococcus pneumoniae, Streptococcus pyogenes, Ureaplasma gallorale, Corynebacterium pyogenes, Pasteuria ramosa, Actinobaccilus seminis, Mycoplasma bovigenitalium, Aspergillus fumigatus, Absidia ramosa, Trypanosoma equiperdum, Babesia caballi, Clostridium tetani or Clostridium botulinum.

34. The method of any one of claims 26 to 31, wherein the target cell is a cancer cell.

35. The method of claim 34, wherein the cancer cell is from a cancer of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus.

36. The method of any one of claims 26 to 31, wherein the target cell is an immune cell.

37. The method of claim 36, wherein the immune cell is a B cell, a T cell, a natural killer cell, or a myeloid cell.

38. The method of any one of claims 26 to 37, wherein the target cell or virus is fluorescently labeled prior to the step (d) of claim 26.

39. The method of any one of claims 26 to 38, wherein the target cell or virus expresses a fluorescent protein.

40. The method of claim 38, wherein the target cell or virus is fluorescently labeled with a dye, a probe, or an antibody in a way that the activity of the target cell is measured by the level of the fluorescence.

41. The method of any one of claims 26 to 40, wherein the predetermined number of target cells is no more than one target cell, or the predetermined number of target viruses is no more than one target virus.

42. The method of any one of claims 26 to 41, wherein the microwell is detected by fluorescent microscopy.

43. A method for identifying a microbe that inhibits replication or survival of a microbial cell, the method comprising:
(a) forming a test sample comprising a plurality of microbial cells, a mixed microbial population, a set of barcoded primers comprising a universal priming sequence, a unique barcode and a universal microbial sequence, and a plurality of universal primers specific for the universal priming sequence;
(b) distributing the test sample into microwells such that each microwell contains no more than one microbe from the mixed microbial population, a predetermined number of microbial cells, no more than 1 barcoded primer, and a plurality of universal primers;
(c) incubating the microwells under conditions and for a period of time sufficient to detect a predetermined amount of replication or survival of the microbial cells in the absence of a microbe;
(d) performing an emulsion PCR reaction to generate barcode-labeled amplification product, wherein each unique barcode is associated with a single microwell in which the amplification took place;
(e) performing next-generation sequencing analysis of the amplification products to identify microbial sequences from droplets in which the microbial cell had a different amount of replication or survival from the predetermined amount.

44. The method of claim 43, wherein the mixed microbial population is a microbiome.

45. The method of claim 44, wherein the microbiome is the microbiome of a wild animal.

46. The method of claim 45, wherein the wild animal is a mammal, a bird, a reptile, a fish, an amphibian, or an invertebrate.

47. The method of any one of claims 43 to 46, wherein the microbial cell is a bacterial pathogen.

48. The method of claim 47, wherein the bacterial pathogen is Acinetobacter baumannii, Neisseria gonorrhea, Neisseria meningitidis, Mycobacterium tuberculosis, Candida albicans, Candida tropicalis, Trichomonas vaginalis, Haemophilus vaginalis, Group B Streptococcus sp., Microplasma hominis, Mycoplasma adleri, Dermatophilus congolensis, Diplorickettsia massiliensis, Mycoplasma agalactiae, Mycoplasma amphoriforme, Mycoplasma fermentans, Mycoplasma genitalium, Mycoplasma haemofelis, Mycoplasma hominis, Mycoplasma hyopneumoniae, Mycoplasma hyorhinis, Mycoplasma pneumoniae, Hemophilus ducreyi, Klebsiella pneumoniae, Granuloma inguinale, Lymphopathia venereum, Treponema pallidum, Mycobacterium tuberculosis, Brucella abortus. Brucella melitensis, Brucella suis, Brucella canis, Campylobacter fetus, Campylobacter fetus intestinalis, Leptospira pomona, Peptostreptococcus anaerobius, Peptostreptococcus asaccharolyticus, Listeria monocytogenes, Staphylococcus aureus, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Streptococcus pneumoniae, Streptococcus pyogenes, Ureaplasma gallorale, Corynebacterium pyogenes, Pasteuria ramosa, Actinobaccilus seminis, Mycoplasma bovigenitalium, Aspergillus fumigatus, Absidia ramosa, Trypanosoma equiperdum, Babesia caballi, Clostridium tetani or Clostridium botulinum.

49. The method of any one of claims 43 to 48, wherein the predetermined number of microbial cells is no more than one microbial cell.

50. The method of any one of claims 43 to 49, wherein the predetermined amount of replication is at least a 2-fold expansion.

51. A method for identifying a microbe that modulates an enzymatic reaction of a target substrate, the method comprising:
(a) forming a test sample comprising a plurality of target substrates and a mixed microbial population;
(b) forming droplets from the test sample such that each droplet contains no more than one microbe from the mixed microbial population and a predetermined amount of target substrates;
(c) incubating the droplets under conditions and for a period of time sufficient to detect a predetermined amount of enzymatic reaction of the target substrate in the absence of a microbe;
(d) isolating a droplet in which the target substrate underwent a different amount of enzymatic reaction from the predetermined amount; and
(e) sequencing DNA from the microbe in the isolated droplet to identify the microbe.

52. The method of claim 51, wherein the target substrate comprises protein, peptide, DNA, RNA, or small molecule compound.

53. The method of claim 51 or 52, wherein the mixed microbial population is a microbiome.

54. The method of claim 53, wherein the microbiome is the microbiome of a wild animal.

55. The method of claim 54, wherein the wild animal is a mammal, a bird, a reptile, a fish, an amphibian, or an invertebrate.

56. The method of any one of claims 51 to 55, wherein the target substrate is fluorescently labeled prior to the step (d) of claim 51.

57. The method of any one of claims 51 to 55, wherein the target substrate is linked to a fluorescent protein.

58. The method of claim 56, wherein the target substrate is fluorescently labeled with a dye, a probe, or an antibody in a way that the enzymatic reaction of the target substrate is measured by the level of the fluorescence.

59. The method of any one of claims 51 to 58, wherein the droplet is isolated by FACS.

60. A method for identifying a microbe that modulates an enzymatic reaction of a target substrate, the method comprising:
(a) forming a test sample comprising a plurality of target substrates and a mixed microbial population;
(b) distributing the test sample into microwells such that each microwell contains no more than one microbe from the mixed microbial population and a predetermined amount of target substrates;
(c) incubating the microwells under conditions and for a period of time sufficient to detect a predetermined amount of enzymatic reaction of the target substrate in the absence of a microbe;
(d) detecting a microwell in which the target substrate underwent a different amount of enzymatic reaction from the predetermined amount; and
(e) sequencing DNA from the microbe in the detected microwell to identify the microbe.

61. The method of claim 60, wherein the target substrate comprises protein, peptide, DNA, RNA, or small molecule compound.

62. The method of claim 60 or 61, wherein the mixed microbial population is a microbiome.

63. The method of claim 62, wherein the microbiome is the microbiome of a wild animal.

64. The method of claim 63, wherein the wild animal is a mammal, a bird, a reptile, a fish, an amphibian, or an invertebrate.

65. The method of any one of claims 60 to 64, wherein the target substrate is fluorescently labeled prior to the step (d) of claim 61.

66. The method of any one of claims 60 to 65, wherein the target substrate is linked to a fluorescent protein.

67. The method of claim 65, wherein the target substrate is fluorescently labeled with a dye, a probe, or an antibody in a way that the enzymatic reaction of the target substrate is measured by the level of the fluorescence.

68. The method of any one of claims 60 to 67, wherein the microwell is detected by fluorescent microscopy.

69. The method of any one of claims 1 to 68, further comprising culturing the identified microbe.

70. The method of any one of claims 1 to 69, further comprising obtaining the mixed microbial population.

71. The method of claim 70, wherein the mixed microbial population is obtained from the microbiome of a wild animal.

72. The method of claim 71, wherein the wild animal is a mammal, a bird, a reptile, a fish, an amphibian, or an invertebrate.

73. The method of claim 70, wherein the mixed microbial population is obtained from a soil sample, a plant sample, a sea sample, a fecal sample, an oral sample, a saliva sample, or a skin swab sample.

74. A method for identifying a microbe that protects a host cell against viral infection, the method comprising:
(a) forming a test sample comprising a plurality of host cells, a mixed microbial population and a plurality of target viruses;
(b) forming droplets from the test sample such that each droplet contains no more than one microbe from the mixed microbial population, a predetermined number of target viruses, and a predetermined number of host cells;
(c) incubating the droplets under conditions and for a period of time sufficient to detect a predetermined amount of activity of the host cells in the presence of the target viruses but absence of a microbe;
(d) isolating a droplet in which the host cell had a different amount of activity than the predetermined amount; and
(e) sequencing DNA from the microbe in the isolated droplet to identify the microbe.

75. The method of claim 74, wherein the activity of the host cell is cell death.

76. The method of claim 75, wherein the predetermined amount of cell death is at least a 2-fold reduction in cell number.

77. The method of claim 74, wherein the mixed microbial population is a microbiome.

78. The method of claim 77, wherein the microbiome is the microbiome of a wild animal.

79. The method of claim 78, wherein the wild animal is a mammal, a bird, a reptile, a fish, an amphibian, or an invertebrate.

80. The method of any one of claims 74 to 79, wherein the host cell is fluorescently labeled prior to the step (d) of claim 1.

81. The method of any one of claims 74 to 80, wherein the host cell expresses a fluorescent protein.

82. The method of any one of claims 74 to 80, wherein the host cell is fluorescently labeled with a dye, a probe, or an antibody in a way that the activity of the host cell is measured by the level of the fluorescence.

83. The method of any one of claims 74 to 82, wherein the predetermined number of host cells is no more than one host cell.

84. The method of any one of claims 74 to 83, wherein the predetermined number of target viruses is no more than one virus.

85. The method of any one of claims 74 to 84, wherein the droplet is isolated by FACS.

86. A method for identifying a microbe that protects a host cell against viral infection, the method comprising:
(a) forming a test sample comprising a plurality of host cells, a plurality of target viruses, and a mixed microbial population;
(b) distributing the test sample into microwells such that each microwell contains no more than one microbe from the mixed microbial population, a predetermined number of target viruses, and a predetermined number of host cells;
(c) incubating the microwells under conditions and for a period of time sufficient to detect a predetermined amount of activity of the host cells in the presence of the target viruses but absence of a microbe;
(d) detecting a microwell in which the host cell had a different amount of activity from the predetermined amount; and
(e) sequencing DNA from the microbe in the detected microwell to identify the microbe.

87. The method of claim 86, wherein the activity of the host cell is cell death.

88. The method claim 87, wherein the predetermined amount of cell death is at least a 2-fold reduction in cell number.

89. The method of claim 86, wherein the mixed microbial population is a microbiome.

90. The method of claim 89, wherein the microbiome is the microbiome of a wild animal.

91. The method of claim 90, wherein the wild animal is a mammal, a bird, a reptile, a fish, an amphibian, or an invertebrate.

92. The method of any one of claims 86 to 91, wherein the host cell is fluorescently labeled prior to the step (d) of claim 86.

93. The method of any one of claims 86 to 92, wherein the host cell expresses a fluorescent protein.

94. The method of any one of claims 86 to 92, wherein the host cell is fluorescently labeled with a dye, a probe, or an antibody in a way that the activity of the host cell is measured by the level of the fluorescence.

95. The method of any one of claims 86 to 94, wherein the predetermined number of host cells is no more than one host cell.

96. The method of any one of claims 86 to 95, wherein the microwell is detected by fluorescent microscopy.

97. The method of any one of claims 74 to 96, wherein the microbe inactivated or eliminated the target virus.

98. The method of any one of claims 1 to 97, wherein the mixed microbial population is a human microbiome.

99. The method of claim 98, wherein the human microbiome is a human gut microbiome.
